(19) United States
(12) Patent Application Publication
(10) Pub. No.: US 2021/0388550 A1
(43) Pub. Date: Dec. 16, 2021

(54) NONWOVEN LOOP

(71) Applicant: Kimberly-Clark Worldwide, Inc., Neenah, WI (US)

(72) Inventors: Jeffrey J. Krueger, Roswell, GA (US); Mehdi Gholipour Baradari, Roswell, GA (US); Adrienne M. Williams, Canton, GA (US)

(21) Appl. No.: 17/279,984

(22) PCT Filed: Sep. 26, 2018
(86) PCT No.: PCT/US2018/052933

§ 371 Date: Mar. 25, 2021

(51) Int. Cl. D04H 3/007; D01F 8/06; D04H 3/016; A61F 13/62

(52) U.S. Cl. CPC D04H 3/007; D01F 8/06; D10B 2403/0111; A61F 13/627; D04H 3/016

(57) ABSTRACT

A nonwoven loop material comprising a crimped bi-component fiber in a side-by-side arrangement with a first side and a second side, wherein the first side has a heat of fusion from about 99 to about 105 (J/g) and the second side has a heat of fusion from about 73 to about 86 (J/g); and wherein the first side has a first viscosity and the second side has a second viscosity and the first and second viscosities are within +/−5 (g/10 minutes) of each other; and the second side comprises a polyolefin and a low crystallinity additive.

### TECHNICAL FIELD

The present invention generally relates to the field of nonwoven materials and webs, and processes for manufacturing the same. More specifically, the present invention is related to crimped fiber nonwoven materials useful as a loop material in mechanical attachment systems, such as a hook and loop mechanical attachment system.

### BACKGROUND OF THE INVENTION

Mechanical fastening systems, such as the type referred to as “hook and loop” fastener systems, have become widely used in various consumer and industrial applications. A few examples of such applications include disposable personal care absorbent articles, protective garments, clothing, sporting goods equipment, and a wide variety of others. Typically, such hook and loop fastening systems are employed in situations where a refastenable connection between two or more materials or articles is desired. These mechanical fastening systems have in many cases replaced other conventional devices used for making such refastenable connections, such as safety pins, buttons, buckles, zippers, and the like.

Mechanical fastening systems typically employ two components, a “male” or hook type component and a “female” or loop type component. The hook component usually includes semi-rigid, hook-shaped elements anchored or connected to a base material. The loop component includes a backing material from which loops project. The hook components are designed to engage the loop components, thereby forming mechanical attachments between two. These mechanical attachments function to resist separation of the respective materials or articles.

Because such disposable products are often intended as single-use items to be discarded after a relatively short period of use, sometimes only a few hours, it is important to reduce the overall expense of materials in the design of such products and to reduce manufacturing costs wherever possible. Thus there exists a continuing need for cost-effective loop fastening material for a mechanical fastening system, particularly as such are used in disposable personal care absorbent articles and disposable protective articles.

### BRIEF SUMMARY OF THE INVENTION

One aspect of the subject matter described in this specification can be implemented as a nonwoven loop material comprising crimped bi-component fibers, each fiber in a side-by-side arrangement with a first side and a second side, wherein the first side has a heat of fusion from about 99 to about 105 (J/g) and the second side has a heat of fusion from about 73 to about 86 (J/g); and wherein the first side has a first melt flow rate and the second side has a second melt flow rate and the first and second melt flow rates are within +/−5 (g/10 minutes) of each other; and the second side comprises a polyolefin and a low crystallinity additive.

Another aspect of the subject matter described in this specification can be implemented as a process for making a nonwoven material comprising providing thermoplastic polymer compositions; forming a plurality of molten bi-component fibers from the thermoplastic polymer compositions, wherein each of the bi-component fibers has a first side with a heat of fusion from about 99 to about 105 (J/g) and a second side has a heat of fusion from about 73 to about 86 (J/g); wherein, the first side has a first melt flow rates and the second side has a second melt flow rate and the first and second melt flow rates are within +/−5 (g/10 minutes) of each other; and the second side comprises a polyolefin and a low crystallinity additive.

Yet a further aspect of the subject matter described in this specification can be implemented as a disposable article comprising an inner body contacting side and an outer non-body contacting side, a hook material, and a nonwoven loop material comprising crimped bi-component fibers in a side-by-side arrangement with a first side and a second side, wherein the first side has a heat of fusion from about 99 to about 105 (J/g) and the second side has a heat of fusion from about 73 to about 86 (J/g); and wherein the first side has a first melt flow rate and the second side has a second melt flow rate and the first and second melt flow rates are within +/−5 (g/10 minutes) of each other; and the second side comprises a polyolefin and a low crystallinity additive.

### BRIEF DESCRIPTION OF THE DRAWINGS

FIG. 1A is a representation of a side-by-side bi-component fiber.

FIG. 1B is a high resolution image of crimped bi-component fibers.

FIG. 1C is a second high resolution image of crimped bi-component fibers.

FIG. 2 is a schematic representation of a process and apparatus for producing a nonwoven loop material.

FIG. 3 is a representation of a disposable diaper.

### DEFINITIONS

As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible spatial configurations of the material. These configurations include, but are not limited to isotactic, syndiotactic and random symmetries.

As used herein the term “fibers” refers to both staple length fibers and continuous filaments, unless otherwise indicated.

As used herein the term “monocomponent” fiber refers to a fiber formed from one or more extruders using only one component. Monocomponent fibers are distinct from multicomponent fibers in that they do not comprise multiple substantially constantly positioned distinct zones of different components across the cross-section of the fiber. This is not meant to exclude fibers formed from one polymer to which small amounts (e.g., less than 30% or less than 20% or less than 10%) of additives have been added for color, anti-static properties, lubrication, hydrophilicity, etc.

As used herein the term “bi-component fibers” refers to fibers which have been formed from at least two component polymers, or the same polymer with different properties or additives, extruded from separate extruders but spun together to form one fiber. Bi-component fibers are also sometimes referred to as conjugate fibers or multicomponent fibers. The polymers are arranged in substantially constantly positioned distinct zones across their cross-sections and extend continuously along the length (or at least a portion of the length) of the multicomponent fibers. The configuration of such a bi-component fiber may be, for example, a sheath/core arrangement wherein one polymer is surrounded by another, or may be a side-by-side arrangement or other arrangements as are known in the art. By way of example, bi-component fibers are taught in U.S. Pat. No. 5,108,820 to Kaneko et al., U.S. Pat. No. 5,336,552 to Strack et al., and U.S. Pat. No. 5,382,400 to Pike et al.

As used herein the term “nonwoven web” or “nonwoven material” means a web having a structure of individual fibers or filaments which are interlaid, but not in an identifiable manner as in a knitted or woven fabric. Nonwoven webs have been formed from many processes such as for example, meltblowing processes, spunbonding processes, air-laying processes and carded web processes.

As used herein “spunbond” fibers and “spunbond” nonwoven webs comprise continuous fiber webs formed by extruding a molten thermoplastic material from a plurality of fine, usually circular, capillaries as molten threads into converging high velocity air streams which attenuate the filaments of molten thermoplastic material to reduce their diameter. The educative drawing of the spunbond process also acts to impart a degree of crystallinity to the formed polymeric fibers which provides a web with relatively increased strength. By way of non-limiting example, spunbond fiber nonwoven webs and processes for making the same are disclosed in U.S. Pat. No. 4,340,563 to Appel et al, U.S. Pat. No. 5,382,400 to Pike et al.; U.S. Pat. No. 8,246,898 to Conrad et al., U.S. Pat. No. 8,333,918 to Lennon et al., and so forth.

### DETAILED DESCRIPTION OF THE INVENTION

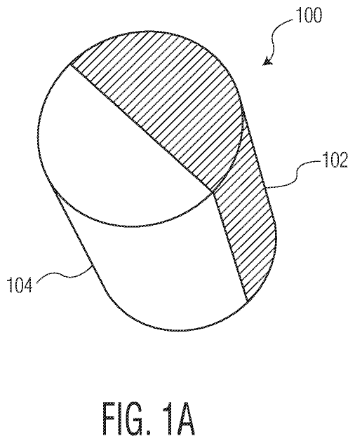

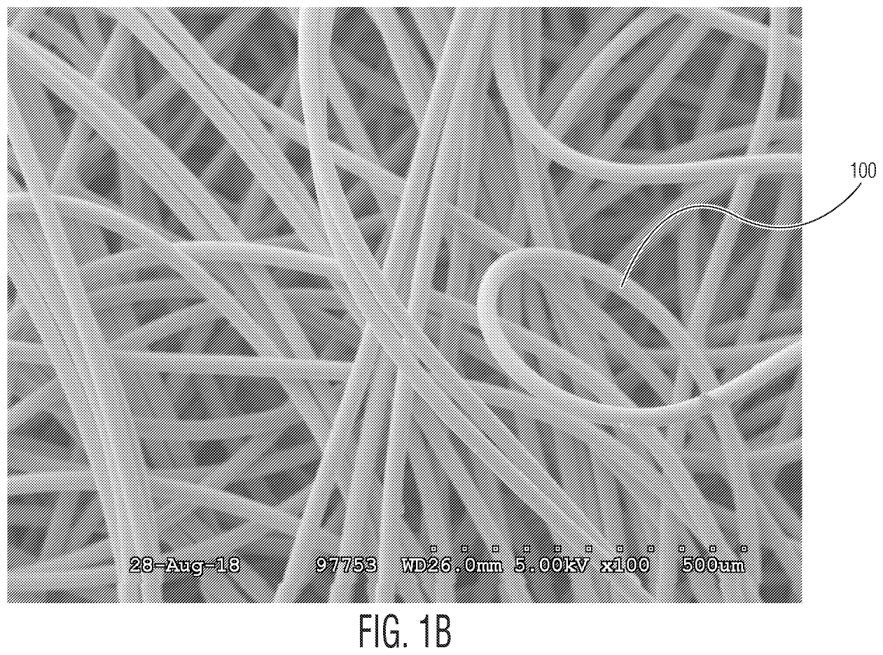

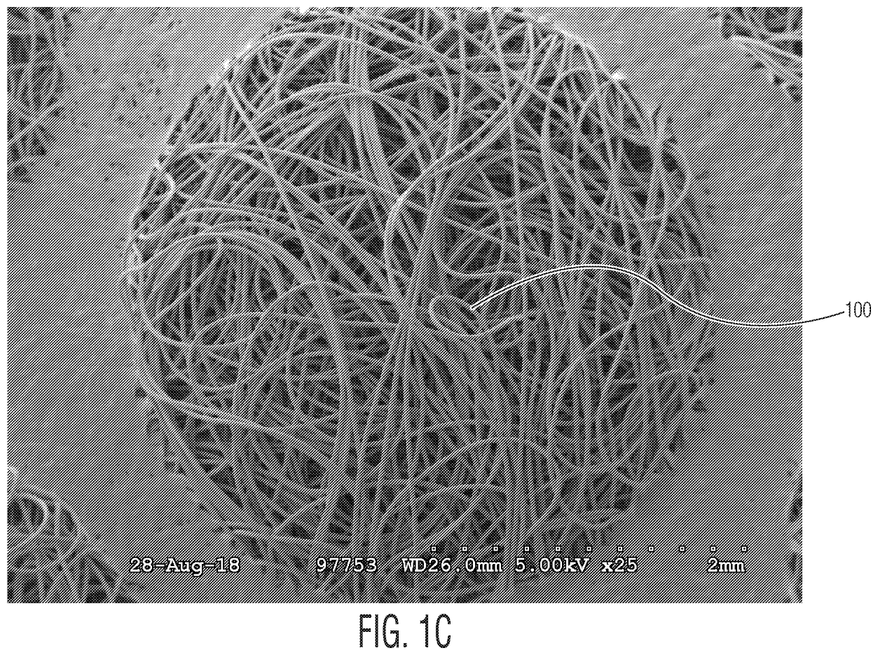

The specification generally describes a nonwoven material suitable as a loop material for a mechanical attachment system, such as a hook and loop mechanical attachment system. More particularly, the loop material is made from side-by-side bi-component fibers having first sides with a heat of fusion from about 99 to about 105 (J/g) and second sides with a heat of fusion from about 73 to about 86 (J/g), melt flow rate differences within +/−5 (g/10 minutes) where the second side is made from a polyolefin and a low crystallinity additive. These properties provide a crimped fiber well suited for use as a loop in a hook and loop fastening system. As described, above, such a crimped fiber is useful for fasteners on personal care articles and garments. Personal care articles include, for example, infant care products such as disposable baby diapers, child care products such as training pants, and adult care products such as incontinence products and feminine care products and garments include, for example, medical apparel, work wear, and the like. The loop material is described in more detail below with reference to FIGS. 1A, 1B and 1C.

As shown in FIG. 1A, the bi-component fiber 100 is arranged in a side-by-side configuration with a first side 102 and a second side 104. In some implementations, the fibers 100 are continuous bi-component filaments with the first side 102 having a first polymeric component A and the second side 104 having a second polymeric component B. For example, the first and second components A and B are arranged in, at least partially, distinct zones across the cross-section of the fiber 100 and extend continuously along the length of the fiber 100. Each of the first and second components A and B, for example, constitute at least a portion of the peripheral surface of the fiber 100 continuously along the length (or a portion of the length) of the fiber 100. As shown in FIGS. 1B and 1C, the fiber 100 is crimped forming a “loop” suitable for, for example, a hook and loop fastening system.

Numerous polymers are suitable to use for the fiber 100. For example, polymer A can be polypropylene and polymer B can be polypropylene-polyethylene copolymer In some implementations, the first side 102 with polymer A has a heat of fusion from about 99 to about 105 (J/g) and more preferably from about 103 to 105, and the second side with polymer B has a heat of fusion from about 65 to about 86 (J/g), preferably from about 73 to about 86 (J/g) and more preferably from about 80 to 86 (J/g) (as measured according to ASTM-D3418). The difference in crystallinity between the first and second sides 102, 104 and their respective polymers A and B, is indicative of the difference of heat of fusion between the first and second sides 102, 104 and their respective polymers A and B. This crystallinity difference contributes to causing the crimp and, thus, the crimp can be at least partially controlled through selection of the crystallinities of the first and second sides 102, 104.

In some implementations, the difference of heat of fusion between the two sides 102, 104 is 8 (J/g) to 35 (J/g), preferably 13 (J/g) to 32 (J/g), more preferably 20 (J/g) to 28 (J/g) and even more preferably 23 (J/g) to 26 (J/g). As described above, careful selection of the crystallinity difference between the first and second sides 102, 104/polymers A and B, along with specified viscosity differences, e.g., within +/−5 (g/10 minutes), results in the crimp of the fiber 100. The viscosity is characterized by melt flow rate (ASTM D1238) with testing conditions of 230° C. and 2.16 kg. In some implementations the first and second sides 102/104 have melt flow rates from about 25 (g/10 min) to 45 (g/10 min) at 230° C. and 2.16 kg.

In some implementations, polymer A from the first side 102 comprises polypropylene, and polymer B from the second side 104 comprises a polyolefin and includes a low crystallinity additive. In some implementations, the first side 102 includes only polypropylene, and the second side 104 includes only a polyolefin and a low crystallinity additive. For example, the polyolefin in the second side 104 can be polypropylene (e.g., 3155 polypropylene available from ExxonMobil) and the low crystallinity additive can be Vistamaxx™ 7050FL available from ExxonMobil or the low crystallinity additive can be L-MODU S400 available from Idemitsu Kosan. A low crystallinity additive is usually soft and flexible (e.g., tensile modulus (ASTMD638) or flexural modulus (ASTMD790) less than about 200 MPa or less than about 100 Mpa) and has a very low heat of fusion (e.g., in some implementations less than 30 J/g and in other implementations less than 25 J/g), and a defined melting point. In some implementations, polymers A and/or B, for sides 102, 104, respectively, may include additives for lowering the bonding temperature of the filaments, and enhancing the abrasion resistance, strength and/or softness of the resulting fabric. Table 1 describes example fiber 100 side compositions.

TABLE 1

| | Heat of Fusion |
|---|---|
| Fiber Side Composition (by % weight) | (J/g) |
| 100% ExxonMobil 3155 PP (e.g., first side 102) | 103 |
| 5% Vistamaxx 7050FL/95% EM3155PP (e.g., first side | 102 |
| 102) | |
| 10% Vistamaxx 7050FL/90% EM3155 PP | 99 |
| 15% Vistamaxx 7050FL/85% EM3155 PP (e.g., second | 93 |
| side 104) | |
| 20% Vistamaxx7050FL/80% EM3155 PP (e.g., second | 86 |
| side 104) | |
| 25% Vistamaxx7050FL/75% EM3155 PP (e.g., second | 77 |
| side 104) | |
| 30% Vistamaxx7050FL/70% EM3155 PP (e.g., second | 65 |
| side 104) | |
| 5% Idemitsu L-MODU S400/95% EM3155 PP(e.g., first | 103 |
| side 102) | |
| 10% Idemitsu L-MODU S400/90% EM3155 PP | 99 |
| 15% Idemitsu L-MODU S400/85% EM3155 PP (e.g., | 94 |
| second side 104) | |
| 20% Idemitsu L-MODU S400/80% EM3155 PP (e.g., | 88 |
| second side 104) | |

In some implementations the fiber 100 diameter is between 19 to about 21 microns, has an air permeability between 440 and 480 cm3/s/cm2 (e.g., per ASTM-D737), and/or has a bulk between 0.57 and 0.63 mm (e.g., per ASTM-D1777).

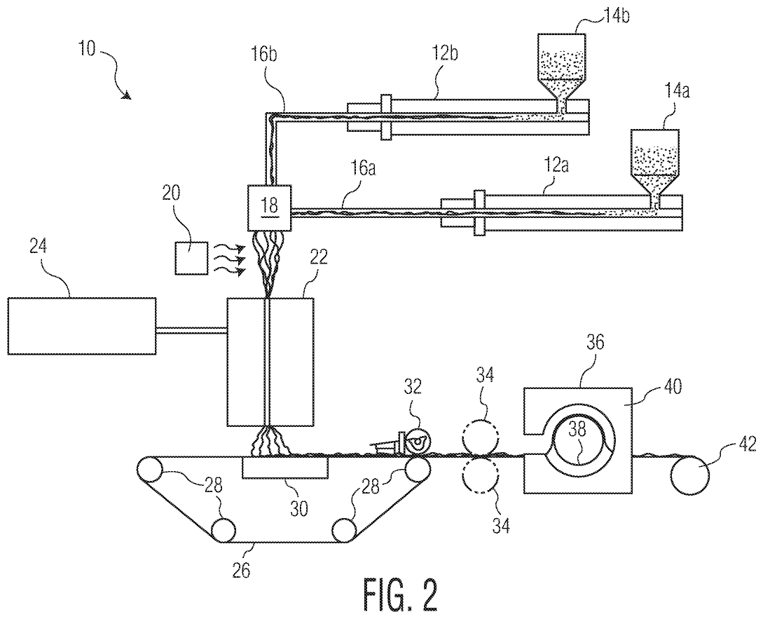

FIG. 2 is a schematic representation of a process and apparatus for producing a nonwoven loop material including fibers 100. The process line 10 is arranged to produce bi-component continuous filaments/fibers 100. The process line 10 includes a pair of extruders 12a and 12b for separately extruding a polymer A (e.g., for the first side 102) and a polymer B (e.g., and the low crystallinity additive for the second side 104). Polymer A is fed into the respective extruder 12a from a first hopper 14a and polymer B is fed into the respective extruder 12b from a second hopper 14b. Polymers A and B are fed from the extruders 12a and 12b through respective polymer conduits 16a and 16b to a spinneret 18.

Generally described, the spinneret 18 includes a housing containing a spin pack which includes a plurality of plates stacked one on top of the other with a pattern of openings arranged to create flow paths for directing polymers A and B separately through the spinneret 18. The spinneret 18 has openings arranged in one or more rows. The spinneret openings form a downwardly extending curtain of filaments when the polymers are extruded through the spinneret 18. The spinneret 18 is arranged to form side-by-side bi-component fiber 100 illustrated in FIGS. 1A, 1B and 1C.

The process line 10 also includes a quench blower 20 positioned adjacent the curtain of filaments extending from the spinneret 18. Air from the quench air blower 20 quenches the filaments extending from the spinneret 18. The quench air can be directed from one side of the filament curtain as shown in FIG. 2, or both sides of the filament curtain.

A fiber draw unit or aspirator 22 is positioned below the spinneret 18 and receives the quenched filaments. Fiber draw units or aspirators for use in melt spinning polymers are well-known. Fiber draw units 22 for use in this process include, for example, a linear fiber aspirator of the type shown in U.S. Pat. No. 3,802,817 and educative guns of the type shown in U.S. Pat. Nos. 3,692,618 and 3,423,266.

Generally described, the fiber draw unit 22 includes an elongate vertical passage through which the filaments/fibers 100 are drawn by aspirating air entering from the sides of the passage and flowing downwardly through the passage. A heater 24 optionally supplies hot aspirating air to the fiber draw unit 22. The hot aspirating air draws the filaments and ambient air through the fiber draw unit 22.

An endless foraminous forming surface 26 is positioned below the fiber draw unit 22 and receives the continuous filaments from the outlet opening of the fiber draw unit 22. The forming surface 26 travels around guide rollers 28. In some implementations, a vacuum 30 is positioned below the forming surface 26 where the filaments are deposited draws the filaments against the forming surface 26.

The process line 10 includes, in some implementations, a compression roller 32 which, along with the forward most of the guide rollers 28, receive the loop material as the web is drawn off of the forming surface 26. In addition, the process line 10 may include, for example, a bonding apparatus such as thermal point bonding rollers 34 or a through-air bonder 36. Generally described, the through-air bonder 36 includes a perforated roller 38, which receives the loop material, and a hood 40 surrounding the perforated roller 38. In some implementations, the process line 10 includes a winding roll 42 for taking up the finished fabric of loop material.

To operate the process line 10, the hoppers 14a and 14b are filled with the respective polymers A and B (and low crystallinity additive). Polymers A and B are melted and extruded by the respective extruders 12a and 12b through polymer conduits 16a and 16b and the spinneret 18. Although the temperatures of the molten polymers vary depending on the polymers used, when polypropylene and polypropylene/low crystallinity additive are used as components A and B respectively, the preferred temperatures of the polymers range from about 400° to about 480° F. and preferably range from 430° to about 450° F.

As the extruded filaments extend below the spinneret 18, a stream of air from the quench blower 20 at least partially quenches the filaments to develop a crimp in the filaments. The quench air preferably flows in a direction substantially perpendicular to the length of the filaments at a temperature of about 40° to about 70° F.

After quenching, the filaments 100 are drawn into the vertical passage of the fiber draw unit 22 by a flow of hot air from the heater 24 through the fiber draw unit 22. The fiber draw unit 22 is, for example, positioned 30 to 60 inches below the bottom of the spinneret 18. The crimped filaments 100 are deposited through the outlet opening of the fiber draw unit 22 onto the traveling forming surface 26.

The vacuum 20 draws the filaments 100 against the forming surface 26 to form an unbonded, nonwoven web of continuous filaments. In some implementations, the web is then lightly compressed by the compression roller 32 and then thermal point bonded by rollers 34 or through-air bonded in the through-air bonder 36. In such implementations, in the through-air bonder 36, air having a temperature above the melting temperature of one of the components but not the other is directed from the hood 40, through the web, and into the perforated roller 38. The hot air melts the lower melting polymer and thereby forms bonds between the bi-component filaments 100 to integrate the web. In some implementations, the loop material is wound onto the winding roller 42 and is ready for further treatment or use.

The nonwoven loop material described herein is useful, for example, in a mechanical attachment system in a wide variety of disposable personal care absorbent articles and disposable protective articles. Disposable personal care absorbent articles include but are not limited to infant and child care absorbent articles such as diapers and training pants, disposable swimwear, adult care incontinent garments, feminine care articles such as sanitary napkins, bandages and wound dressings, and the like. Disposable protective articles include but are not limited to such articles as surgical gowns and surgical drapes, patient examination gowns, industrial workwear and cleanroom apparel. Such disposable personal care and protective articles generally have a body facing side which is worn or placed against or towards the body of the user and a non-body facing side facing away from the body of the user.

The nonwoven loop material, when used as part of a mechanical attachment system for such disposable personal care and protective articles, would generally be placed on or attached to the outer or non-body facing side of the article, or alternatively the outer or non-body facing side of the article may be composed wholly of the nonwoven loop material of the invention. The hook material would generally be placed on or comprise a tab on the article which is conveniently located on the article such that the user or wearer is able to superimpose the hook tab in face to face relation with the loop material, such that hook components can engage the fibers of the loop material.

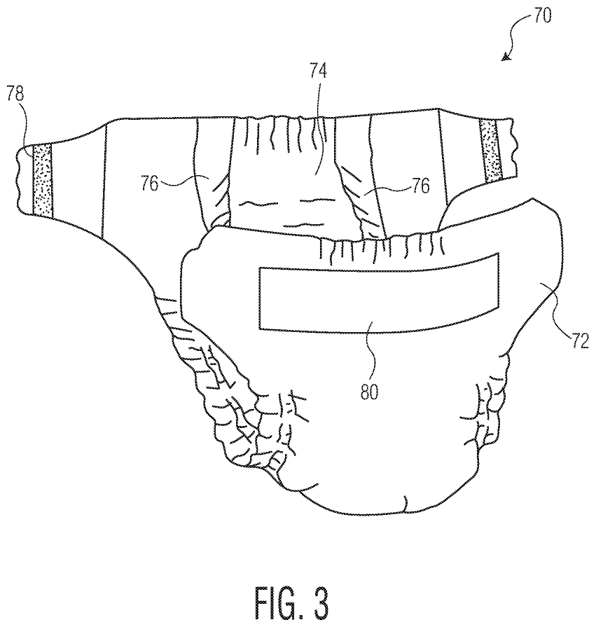

With reference to FIG. 3, there is shown an example personal care article such as the diaper 70. The diaper 70, as is typical for most personal care absorbent articles, includes a liquid permeable body side liner 74, i.e., a body-facing or inner side, and a liquid impermeable outer cover 72, i.e., a non-body facing or outer side. Various woven or nonwoven fabrics can be used for body side liner 74 such as a spunbond nonwoven web of polyolefin fibers, or a bonded carded web of natural and/or synthetic fibers. In some implementations, the outer cover 72 is formed of a thin liquid barrier material such as for example a spunbond-meltblown layer, spunbond-meltblown-spunbond layer, or a thermoplastic polymer film layer. A polymer film outer cover may be embossed and/or matte finished to provide a more aesthetically pleasing appearance, or may be a laminate formed of a woven or nonwoven fabric and thermoplastic film. The outer cover 72 may optionally be composed of a “breathable” material that is permeable to vapors or gas yet substantially impermeable to liquid. Examples of outer cover materials include but are not limited to those disclosed in U.S. Pat. No. 6,309,736 to McCormack et al., the disclosure of which is incorporated herein by reference in its entirety.

Disposed between liner 74 and outer cover 72 is an absorbent core (not shown) formed, for example, of a blend of hydrophilic cellulosic wood pulp fluff fibers and highly absorbent gelling particles (e.g., superabsorbent material). The diaper 70 may further include optional containment flaps 76 made from or attached to body side liner 74. Suitable constructions and arrangements for such containment flaps are described, for example, in U.S. Pat. No. 4,704,116 to Enloe, the disclosure of which is incorporated herein by reference in its entirety. Still further, the diaper 70 can optionally include, but not limited to, elasticized leg cuffs, elastic waist band, and so forth.

To secure the diaper 70 about the wearer, the diaper 70 will have a fastening system. As shown in FIG. 3, the fastening system is a hook and loop fastening system including hook elements 78 attached to the inner and/or outer surface of outer cover 72 in the back waistband region of diaper 70 and one or more loop elements or patches 80 made from the nonwoven loop material, including the fibers 100, attached to the outer surface of outer cover 72 in the front waistband region of diaper 70. The nonwoven loop material (with fibers 100) can be secured to outer cover 72 of diaper 70 by known attachment means, including but not limited to adhesives, thermal bonding, ultrasonic bonding, or a combination of such means. As an alternative embodiment, the nonwoven loop material may cover substantially all or all of the outer surface of outer cover 72. An example of this would be an outer cover material constructed of a thermoplastic film/nonwoven loop material laminate.

1. A nonwoven loop material comprising:
a crimped bi-component fiber in a side-by-side arrangement with a first side and a second side, wherein the first side has a heat of fusion from about 99 to about 105 (J/g) and the second side has a heat of fusion from about 73 to about 86 (J/g); and
wherein:
the first side has a first melt flow rate and the second side has a second melt flow rate and the first and second melt flow rates are within +/−5 (g/10 minutes) of each other; and
the second side comprises a polyolefin and a low crystallinity additive.

2. The nonwoven loop material of claim 1, wherein the polyolefin is polypropylene.

3. The nonwoven loop material of claim 1, wherein the first side comprises a polyolefin.

4. The nonwoven loop material of claim 1, wherein the nonwoven loop material has a fiber diameter from about 18 to about 31 microns.

5. The nonwoven loop material of claim 1, wherein the nonwoven loop material has an air permeability of about 440 to about 480 cm3/s/cm2.

6. The nonwoven material of claim 1, wherein the crimped bi-component fibers are continuous fibers.

7. The nonwoven material of claim 1, wherein the nonwoven loop material has a bulk from about 0.025 to about 0.035 mm.

8. A process for making a nonwoven material comprising:
providing thermoplastic polymer compositions;
forming a plurality of molten bi-component fibers from the thermoplastic polymer compositions, wherein each of the bi-component fibers has a first side with a heat of fusion from about 99 to about 105 (J/g) and a second side has a heat of fusion from about 73 to about 86 (J/g); wherein, the first side has a first melt flow rate and the second side has a second melt flow rate and the first and second melt flow rates are within +/−5 (g/10 minutes) of each other; and the second side comprises a polyolefin and a low crystallinity additive.

9. The process for making a nonwoven material of claim 8, wherein the polyolefin is polypropylene.

10. The process for making a nonwoven material of claim 8, wherein the first side comprises a polyolefin.

11. The process for making a nonwoven material of claim 8, wherein the nonwoven loop material has a fiber diameter from about 18 to about 31 microns.

12. The process for making a nonwoven material of claim 8, wherein the nonwoven loop material has an air permeability of about 440 to about 480 cm3/s/cm2.

13. The process for making a nonwoven material of claim 8, wherein the crimped bi-component fibers are continuous fibers.

14. The process for making a nonwoven material of claim 8, wherein the nonwoven loop material has a bulk from about 0.025 to about 0.035 mm.

15. A disposable article comprising:
an inner body contacting side and an outer non-body contacting side,
a hook material, and
a nonwoven loop material comprising crimped bi-component fibers in a side-by-side arrangement with a first side and a second side, wherein the first side has a heat of fusion from about 99 to about 105 (J/g) and the second side has a heat of fusion from about 73 to about 86 (J/g); and wherein the first side has a first melt flow rate and the second side has a second melt flow rate and the first and second melt flow rates are within +/−5 (g/10 minutes) of each other; and the second side comprises a polyolefin and a low crystallinity additive.

16. The disposable article of claim 15, wherein the polyolefin is polypropylene.

17. The disposable article of claim 15, wherein the first side comprises a polyolefin.
